(19) United States
(12) Patent Application Publication
(10) Pub. No.: US 2005/0148647 A1
(43) Pub. Date: Jul. 7, 2005

(54) Hemodialysis solutions and uses thereof

(76) Inventor: Donald W. Landry, New York, NY (US)

Correspondence Address: COOPER & DUNHAM, LLP, 1185 AVENUE OF THE AMERICAS, NEW YORK, NY, 10036, US

(21) Appl. No.: 10/976,733

(22) Filed: Oct. 29, 2004

Related U.S. Application Data
(60) Provisional application No. 60/516,667, filed on Oct. 31, 2003.

(51) Int. Cl. A61K 31/4172; A61K 31/401; A61K 31/198; A61K 31/405

(52) U.S. Cl. 514/400; 514/423; 514/561; 514/562; 514/563; 514/565; 514/419

(57) ABSTRACT

This invention provides hemodialysis solutions comprising certain amino acids in certain pre-determined amounts. This invention also provides methods and kits for performing dialysis in a manner which treats or inhibits the onset of malnutrition in the dialysis patient.

This application claims priority of U.S. Ser. No. 60/516,667, filed Oct. 31, 2003, the contents of which are incorporated herein by reference.

Throughout this application, various publications are referenced by author and publication date. Full citations for these publications may be found at the end of the specification immediately preceding the claims. The disclosures of these publications are hereby incorporated by reference into this application to describe more fully the art to which this invention pertains.

### BACKGROUND OF THE INVENTION

Malnutrition is a common problem among patients with end-stage renal disease (ESRD) maintained on either chronic hemodialysis or peritoneal dialysis. The estimated prevalence of malnutrition in patients with ESRD ranges from 40 to 70%, depending on which markers of nutritional status are used (Rocco et al. 2002). This level of malnutrition is difficult to tolerate given that lean-body wasting and protein calorie malnutrition are the major predictors of morbidity and mortality among ESRD patient populations (Pupim et al. 2002).

The malnourished state of ESRD is often attributed to anorexia, a decreased sense of taste, phosphate binders that prevent proper nutrient absorption, among other poorly quantifiable factors. However, it is also clear that both hemodialysis and peritoneal dialysis result in a dramatic loss of protein and amino acids, up to 6-10 grams per day, which can only exacerbate a patient's already marginal state of nutrition (Hynote et al. 1995). Dialysis leaves the body in a catabolic state, vastly worsening net protein metabolism and malnutrition (Teschner et al. 1989). ESRD itself leads to muscle wasting and an eventual poor outcome, but the process of dialysis adds an additional insult to protein metabolism that providers would address if a financially feasible therapy were available.

Malnutrition in ESRD is a potentially reversible condition, and efforts have been focused on replacing or minimizing the nutritional losses incurred during dialysis. Several studies have demonstrated a benefit from intradialytic parenteral nutrition (IDPN), which entails the infusion of nutrients through a venous line during the dialysis treatment. The benefit likely accrues from replacing protein and amino acids lost during the process of dialysis treatment. One study showed retrospectively that after nine months, patients given IDPN had statistically significantly higher serum albumin and an apparent improvement in survival over the control group with no IDPN (Capelli et al. 1994). Another study demonstrated through direct arterial and venous measurements of metabolic markers that the forearm muscle of patients with IDPN was converted from a catabolic state to an anabolic state (Pupim et al. 2002). Though large, randomized controlled trials are lacking, the suggested benefit is compelling and the United States government will reimburse for IDPN in sufficiently malnourished ESRD patients.

IDPN, however, suffers from a number of problems. Only 70% of the intravenous grade nutrients are delivered to the patient due to substantial losses of costly amino acids into the dialysate. In fact, IDPN costs twice as much as dialysis itself, which is a tremendous barrier to overcome in treating ESRD patients with malnutrition (Wolfson et al. 1982). Of note, the clinical threshold for Medicare reimbursement is very severe malnutrition; the majority of malnourished ESRD patients is ineligible for IDPN and remains untreated.

Alternative methods by which to deliver the necessary nutrients to dialysis patients have been the subject of investigation. Compared to IDPN, the addition of amino acids to the dialysate is no less efficient at maintaining plasma concentrations of amino acids. With an elevated concentration of amino acids in dialysate, there is a lower gradient for removal across the membrane. Potentially, with high enough dialysate concentrations that reverse the gradient, one could use this method to boost plasma amino acid concentrations with each dialysis treatment.

In 1978, a method was descibed in which amino acids were added directly to the dialysate roughly in proportion to normal plasma concentrations in order to minimize their loss during dialysis (Quarto di Palo et al. 1978). More recently, the effectiveness of this procedure was further evaluated by randomizing patients to dialysate with (1) no amino acids, (2) amino acids in the dialysate that were equal to normal plasma concentration, or (3) amino acids in the dialysate three times normal plasma concentration (Chazot et al. 1997). The results demonstrated a positive net balance of amino acids when the dialysate was enriched and a net loss without the addition of amino acids. The post-dialytic plasma concentrations were also substantially higher with the 3 times normal plasma as opposed to simple 1× plasma concentration.

Thus, the addition of amino acids directly to the dialysate would be an ideal method to treat protein calorie malnutrition if the amino acids could be obtained easily and inexpensively in large quantities. However, for both the IDPN and the amino acid dialysate modalities, the acquisition and utilization of amino acids is a cumbersome and costly process. In current procedures, the amino acids are purified from hydrolyzed proteins, separated and prepared as sterile solutions which are then recombined in proportions to match normal plasma concentrations.

### SUMMARY OF THE INVENTION

This invention provides a hemodialysis solution comprising amino acids in amounts approximately proportional to that of amino acids obtained from the hydrolysis of casein.

This invention also provides a hemodialysis solution comprising amino acids in amounts approximately proportional to that of amino acids obtained from the hydrolysis of whey or lactalbumin.

This invention further provides a hemodialysis solution comprising amino acids, wherein the amino acids are derived from the hydrolysis of soy protein.

This invention also provides a method for performing dialysis on a subject in a manner which inhibits the loss of amino acids from the subject during dialysis, comprising performing dialysis on the subject using a dialysis solution of the instant invention.

This invention also provides a method for performing dialysis on a malnourished subject in a manner which permits treatment of the malnutrition, comprising performing dialysis on the subject using a dialysis solution of the instant invention, wherein for a majority of the amino acids in the dialysis solution, the concentration of each such amino acid is greater than its respective concentration in a normal subject's blood.

This invention also provides a method for performing dialysis on a subject in a manner which inhibits the onset of malnutrition in the subject, comprising performing dialysis on the subject using a dialysis solution of the instant invention.

This invention also provides a method for performing dialysis on a subject in a manner which permits the introduction of amino acids into the subject, comprising performing dialysis on the subject using a dialysis solution of the instant invention, wherein for a majority of the amino acids in the dialysis solution, the concentration of each such amino acid is greater than its respective concentration in a normal subject's blood.

This invention also provides a kit for use in performing dialysis on a subject comprising (a) a dialysis solution of the instant invention, and (b) instructions for using the solution in performing dialysis in order to inhibit amino acid loss from the subject, treat malnutrition in the subject, inhibit the onset of malnutrition in the subject, and/or introduce amino acids into the subject, wherein for a majority of the amino acids in the dialysis solution, the concentration of each such amino acid is greater than its respective concentration in a normal subject's blood.

Finally, this invention provides a kit for use in performing hemodialysis on a subject comprising (a) amino acids in the relative amounts which would result from the hydrolysis of casein, whey, lactalbumin, or soy protein, and (b) instructions for using the amino acids in performing hemodialysis in order to inhibit amino acid loss from the subject, treat malnutrition in the subject, inhibit the onset of malnutrition in the subject, and/or introduce amino acids into the subject.

### BRIEF DESCRIPTION OF THE FIGURES

FIG. 1: Normal plasma concentrations of amino acids shown in micromoles per liter. Essential amino acids are indicated by an asterisk.

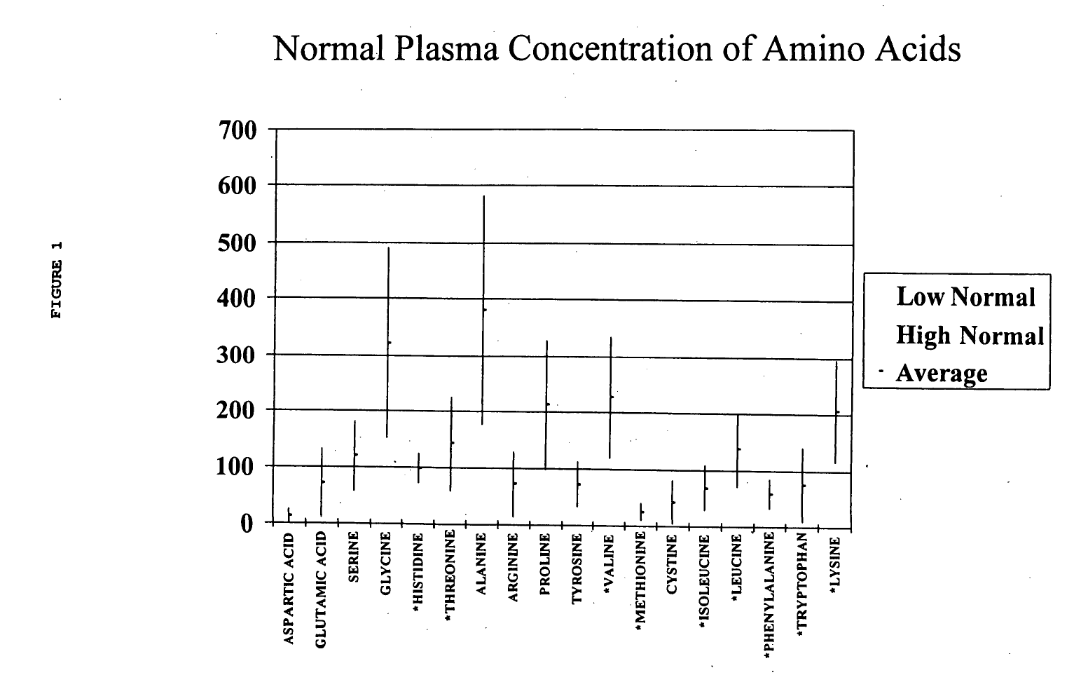

### DETAILED DESCRIPTION OF THE INVENTION

The instant invention solves the problems of identifying ideal amino acid compositions for use in dialysis and identifying a cost-effective source of such amino acids for that use. This invention thus represents a new and useful improvement in the art of nutrition for dialysis patients. Rather than separate, purify and recombine individual amino acids, the present invention, in a preferred embodiment, utilizes the protein casein which, upon hydrolysis, yields essential amino, acids in appropriate proportions for immediate use in the supplementation of dialysate. Indeed, casein contains all nine essential amino acids and nine additional nonessential amino acids. Any protein that could be obtained cheaply and in large quantities which has an amino acid content similar to that of casein could be used in its place.

Specifically, this invention provides a hemodialysis solution comprising amino acids, wherein (a) the identity of each amino acid present in the solution and (b) the approximate amount, by weight, of each amino acid in the solution, expressed as the ratio of the approximate amount of the amino acid relative to the approximate amount of tryptophan present therein, are as follows:

| amino acid | amount |
|---|---|
| alanine | 2.5 |
| arginine | 3.3 |
| aspartate | 5.7 |
| cysteine | 0.3 |
| glutamate | 19.2 |
| glycine | 2.0 |
| histidine | 2.5 |
| isoleucine | 5.1 |
| leucine | 7.6 |
| lysine | 6.8 |
| methionine | 2.5 |
| phenylalanine | 4.2 |
| proline | 9.0 |
| serine | 5.1 |
| threonine | 3.8 |
| tryptophan | 1.0 |
| tyrosine | 5.5 |
| valine | 4.5 |

In an embodiment of the invention, the amino acids are derived from the hydrolysis of a protein. The hydrolysis can be an acid hydrolysis or an enzymatic hydrolysis. In one embodiment, the protein is casein.

For example, in one embodiment, the solution comprises amino acids in the following amounts:

| amino acid | amount (g/L) |
|---|---|
| alanine | 2.8 |
| arginine | 3.6 |
| aspartate | 6.3 |
| cysteine | 0.3 |
| glutamate | 21.1 |
| glycine | 2.2 |
| histidine | 2.7 |
| isoleucine | 5.6 |
| leucine | 8.4 |
| lysine | 7.5 |
| methionine | 2.7 |
| phenylalanine | 4.6 |
| proline | 9.9 |
| serine | 5.6 |
| threonine | 4.2 |
| tryptophan | 1.1 |
| tyrosine | 6.1 |
| valine | 5 |

These amounts can be obtained from the hydrolysis of casein. Hydrolyzed casein is available from commercial sources at low cost. One such source is the American Casein Company, of Burlington, N.J.

Casein can be obtained from any mammalian source. In one embodiment the casein is bovine casein.

This invention also provides a hemodialysis solution comprising amino acids, wherein (a) the identity of each amino acid present in the solution and (b) the approximate amount, by weight, of each amino acid in the solution, expressed as the ratio of the approximate amount of the amino acid relative to the approximate amount of tryptophan present therein, are as follows:

| amino acid | amount |
|---|---|
| alanine | 3.6 |
| arginine | 1.9 |
| aspartate | 7.4 |
| cysteine | 1.2 |
| glutamate | 12.3 |
| glycine | 1.5 |
| histidine | 1.4 |
| isoleucine | 4.7 |
| leucine | 7.6 |
| lysine | 6.5 |
| methionine | 1.6 |
| phenylalanine | 2.2 |
| proline | 4.6 |
| serine | 3.6 |
| threonine | 3.5 |
| tryptophan | 1.0 |
| tyrosine | 2.5 |
| valine | 3.3 |

In an embodiment of the invention, the amino acids are derived from the hydrolysis of a protein. The hydrolysis can be an acid hydrolysis or an enzymatic hydrolysis. In an embodiment, the protein is whey or lactalbumin.

This invention further provides a hemodialysis solution comprising amino acids, wherein the amino acids are derived from the hydrolysis of soy protein.

In an embodiment of any of the hemodialysis solutions of this invention, the solution further comprises sodium, chloride, bicarbonate, lactose, calcium and/or magnesium. In an embodiment, the pH of the solution is approximately 6.9-7.4.

In an embodiment of any of the hemodialysis solutions of this invention, the solution further comprises potassium and/or glucose. For example, a hemodialysis solution of the instant invention comprises amino acids as set forth in this invention and the following: sodium, 140 mmol/L; potassium, 2 mmol/L; calcium, 3 mmol/L; bicarbonate, 39 mmol/L; and chloride 108 mmol/L. In another embodiment, the solution further comprises glucose, 1 g/L.

This invention also provides a method for performing dialysis on a subject in a manner which inhibits the loss of amino acids from the subject during dialysis, comprising performing dialysis on the subject using a dialysis solution of the instant invention.

In one embodiment of the dialysis solution, for a majority of the amino acids in the dialysis solution, the concentration of each such amino acid is at least as great as its respective concentration in a normal subject's blood. In one embodiment, the subject is a human.

This invention also provides a method for performing dialysis on a malnourished subject in a manner which permits treatment of the malnutrition, comprising performing dialysis on the subject using a dialysis solution of the instant invention, wherein for a majority of the amino acids in the dialysis solution, the concentration of each such amino acid is greater than its respective concentration in a normal subject's blood. In one embodiment, the subject is a human.

This invention also provides a method for performing dialysis on a subject in a manner which inhibits the onset of malnutrition in the subject, comprising performing dialysis on the subject using a dialysis solution of the instant invention. In one embodiment, the dialysis solution has amino acid concentrations which are, for a majority of the amino acids, at least equal to the respective amino acid concentrations in a normal subject's blood. In another embodiment, the dialysis solution has amino acid concentrations which are, for a majority of the amino acids, greater than the respective amino acid concentrations in a normal subject's blood. In an embodiment of any of these methods, the subject is a human.

This invention also provides a method for performing dialysis on a subject in a manner which permits the introduction of amino acids into the subject, comprising performing dialysis on the subject using a dialysis solution of the instant invention, wherein for a majority of the amino acids in the dialysis solution, the concentration of each such amino acid is greater than its respective concentration in a normal subject's blood.

This invention also provides a kit for use in performing dialysis on a subject comprising (a) a dialysis solution of the instant invention, and (b) instructions for using the solution in performing dialysis in order to inhibit amino acid loss from the subject, treat malnutrition in the subject, inhibit the onset of malnutrition in the subject, and/or introduce amino acids into the subject, wherein for a majority of the amino acids in the dialysis solution, the concentration of each such amino acid is greater than its respective concentration in a normal subject's blood.

In one embodiment of each of the instant kits, the amino acid concentration of the solution in the kit is from about 25-50 times, or preferably 35 to 45 times, greater than the amino acid concentration in the solution would be once the solution is diluted (e.g., by a dialysis machine) prior to use of the solution in dialysis. In other words, the solution in such kit, in this embodiment, is in the form of a concentrate.

Finally, this invention provides a kit for use in performing hemodialysis on a subject comprising (a) amino acids in the relative amounts which would result from the hydrolysis of casein, whey, lactalbumin, or soy protein, and (b) instructions for using the amino acids in performing hemodialysis in order to inhibit amino acid loss from the subject, treat malnutrition in the subject, inhibit the onset of malnutrition in the subject, and/or introduce amino acids into the subject.

This invention is illustrated in the Experimental Details section which follows. This section is set forth to aid in an understanding of the invention but is not intended to, and should not be construed to, limit in any way the invention as set forth in the claims which follow thereafter.

Experimental Details

Methods

Supplementation with casein hydrolysate was evaluated in a dog dialysis model. Two dogs were dialyzed: a control animal underwent four hours of standard hemodialysis. The second animal was dialyzed with dialysate enriched with vitamin-free casein acid hydrolysate under otherwise identical conditions. The dogs were anesthetized and ventilated; venous access for dialysis was achieved via two peripheral venous lines, right femoral and left external jugular. A routine, bicarbonate-based dialysis solution, Centrisol™, was used with both animals and diluted with filtered water by the hemodialysis machine to the appropriate concentration. For the experimental animal, the initial dialysate was supplemented with 100 grams of casein hydrolysate in one liter of water, which was then diluted by the hemodialysis machine to the appropriate sodium concentration of approximately 137 mEq/L before passing through the Baxter PSN 170 filter. The animals were dialyzed for four hours with a blood flow rate of 300 ml/min and a dialysate flow rate of 500 ml/min. Plasma and dialysate samples were from both animals at t=0 and t=4 hours were analyzed for amino acid content. Additional samples were taken from the serum post-filter and from the dialysate immediately pre-filter.

Results

Results from the two-dog experiment demonstrated an increase in serum concentrations of eight of the nine essential amino acids in the casein group, while the ninth essential amino acid stayed the same in concentration (Table 1). For the control group, three amino acids decreased in serum concentrations, four increased, and two stayed the same. Comparing the increases in essential amino acids between the two groups, the increase in the casein group was consistently substantially larger than any increase in the control group. The average increase in the experimental group was 76.25 micromoles/L compared to 22.67 micromoles/L for the control group. These results show that the casein-enriched dialysate either supported or bolstered every essential amino acid compared to standard dialysis.

TABLE 1

| amino acid | C t1 | C t2 | S t1 | S t2 | C % | S % |
|---|---|---|---|---|---|---|
| alanine | 674 | 704 | 658 | 712 | 4.45 | 8.2 |
| arginine | 174 | 124 | 132 | 148 | −28.74 | 12.1 |
| aspartate | 4 | 4 | 2 | 12 | 0 | 500.0 |
| cysteine | 10 | 10 | 8 | 12 | 0 | 50.0 |
| glutamate | 52 | 32 | 26 | 36 | −38.46 | 38.5 |
| glycine | 292 | 178 | 306 | 260 | −39.1 | −15.0 |
| histidine* | 76 | 70 | 68 | 88 | −7.9 | 29.4 |
| isoleucine* | 30 | 64 | 46 | 138 | 113.33 | 200.0 |
| leucine* | 106 | 156 | 126 | 290 | 47.2 | 130.2 |
| lysine* | 122 | 104 | 98 | 198 | −14.75 | 102.0 |
| methionine* | 42 | 46 | 44 | 86 | 9.524 | 95.5 |
| phenylalanine* | 34 | 56 | 34 | 82 | −64.7 | 141.2 |
| proline | 288 | 182 | 336 | 428 | −36.81 | 27.4 |
| serine | 168 | 100 | 154 | 154 | −40.48 | 0.0 |
| threonine* | 120 | 102 | 108 | 128 | −15 | 18.5 |
| tryptophan* | 44 | 48 | 52 | 52 | 9.09 | 0.0 |
| tyrosine | 40 | 40 | 28 | 48 | 0 | 71.4 |
| valine* | 98 | 120 | 118 | 242 | 22.45 | 105.1 |

Plasma amino acid concentrations (μmol/L) before (t1) and after (t2) dialysis for animal receiving non-supplemented (C) or amino acid supplemented (S) dialysis solution. The percentage change in plasma amino acid concentrations is also shown (right two columns). Essential amino acids are indicated by an asterisk after the name.

The results further demonstrate that of the nine nonessential amino acids, seven were increased in the casein group, as opposed to only one in the control group. (Table 1). Glycine was the only amino acid that decreased, and serine remained the same concentration for the casein group. Five of the amino acids in the control group decreased over the course of dialysis. These data show that the nonessential amino acids in plasma are also supported by the addition of casein to dialysate compared to standard dialysis.

TABLE 2
Amino acid concentrations in supplemented
dialysis solution.

| amino acid | μmol/L |
|---|---|
| alanine | 346 |
| arginine | 118 |
| aspartate | 228 |
| cysteine | 6 |
| glutamate | 346 |
| glycine | 130 |
| histidine | 60 |
| isoleucine | 152 |
| leucine | 374 |
| lysine | 214 |
| methionine | 80 |
| phenylalanine | 84 |
| proline | 698 |
| serine | 200 |
| threonine | 110 |
| tryptophan | 2 |
| tyrosine | 20 |
| valine | 222 |

### Conclusions

In the United States, casein is readily purchased from large volume distributors in a readily useable hydrolyzed form. The constituent amino acids are partially neutralized and suitable for direct addition to dialysate concentrate. The cost savings from the use of hydrolyzed casein supplementation of dialysate, compared to the present practice of IDPN, is sizable with direct consequences to patient care. At present, IDPN is used only when absolutely necessary because of cost, which is estimated to be twice the cost of dialysis itself—not an insignificant cost per patient per year. Notably, casein hydrolysate provides the substantially equivalent benefit at less than 1/500 the cost of IDPN. More malnourished patients could be treated for the present cost, broadening the reach of therapy. The impact on the quality of life of these patients would be enormous.

### References

Capelli J. P. et al. Effect of intradialytic parenteral nutrition on mortality rates in end-stage renal disease care. Am. J. Kidney Dis. 23(6): 808-16 (1994).
Chazot C. et al. Dialystic nutrition: Provision of amino acids in dialysate during hemodialysis, Kidney International 52: 1663-70 (1997).
Hynote E. D. et al. Amino acid losses during hemodialysis: Effects of high-solute flux and parenteral nutrition in acute renal failure, J. Parenteral and Enteral Nutrition 19(1): 15-21 (1995).
Pupim, L. B. et al, Intradialytic parenteral nutrition improves protein and energy homeostasis in chronic hemodialysis patients. J. Clin. Invest. 110: 483-492 (2002).
Quarto di Palo F. et al. Clinical use of a dialysis solution containing amino acids. Intl. J. Artificial Organs 1(1): 112-13 (1978).
Rocco M. V. et al. Nutritional status in the HEMO Study cohort at baseline. Am. J. Kidney Dis. 39(2): 245-56 (2002).
Teschner M. and Heidland A. Hypercatabolism in acute renal failure—Mechanisms and therapeutic approaches. Blood Purif. 7: 16-27 (1989).
Wolfson M. et al. Amino acid losses during hemodialysis with infusion of amino acids and glucose. Kidney Int. 21(3): 500-06 (1982).

1. A hemodialysis solution comprising amino acids, wherein (a) the identity of each amino acid present in the solution and (b) the approximate amount, by weight, of each amino acid in the solution, expressed as the ratio of the approximate amount of the amino acid relative to the approximate amount of tryptophan present therein, are as follows:

| amino acid | amount |
|---|---|
| alanine | 2.5 |
| arginine | 3.3 |
| aspartate | 5.7 |
| cysteine | 0.3 |
| glutamate | 19.2 |
| glycine | 2.0 |
| histidine | 2.5 |
| isoleucine | 5.1 |
| leucine | 7.6 |
| lysine | 6.8 |
| methionine | 2.5 |
| phenylalanine | 4.2 |
| proline | 9.0 |
| serine | 5.1 |
| threonine | 3.8 |
| tryptophan | 1.0 |
| tyrosine | 5.5 |
| valine | 4.5 |

2. The solution of claim 1, wherein the amino acids are derived from the hydrolysis of a protein.

3. The solution of claim 2, wherein the hydrolysis is an acid hydrolysis.

4. The solution of claim 2, wherein the hydrolysis is an enzymatic hydrolysis.

5. The solution of claim 2, wherein the protein is casein.

6. The solution of claim 1, further comprising sodium, chloride, bicarbonate, lactose, calcium and/or magnesium.

7. The solution of claim 1, wherein the pH of the solution is approximately 6.9 to 7.4.

8. A hemodialysis solution comprising amino acids, wherein the amino acids are derived from the hydrolysis of casein.

9. The solution of claim 8, wherein the casein is bovine casein.

10. The solution of claim 8, further comprising sodium, chloride, bicarbonate, lactose, calcium and/or magnesium.

11. The solution of claim 8, wherein the pH of the solution is approximately 6.9 to 7.4.

12. A hemodialysis solution comprising amino acids, wherein (a) the identity of each amino acid present in the solution and (b) the approximate amount, by weight, of each amino acid in the solution, expressed as the ratio of the approximate amount of the amino acid relative to the approximate amount of tryptophan present therein, are as follows:

| amino acid | amount |
|---|---|
| alanine | 3.6 |
| arginine | 1.9 |
| aspartate | 7.4 |
| cysteine | 1.2 |
| glutamate | 12.3 |
| glycine | 1.5 |
| histidine | 1.4 |
| isoleucine | 4.7 |
| leucine | 7.6 |
| lysine | 6.5 |
| methionine | 1.6 |
| phenylalanine | 2.2 |
| proline | 4.6 |
| serine | 3.6 |
| threonine | 3.5 |
| tryptophan | 1.0 |
| tyrosine | 2.5 |
| valine | 3.3 |

13. The solution of claim 12, wherein the amino acids are derived from the hydrolysis of a protein.

14. The solution of claim 13, wherein the hydrolysis is an acid hydrolysis.

15. The solution of claim 13, wherein the hydrolysis is an enzymatic hydrolysis.

16. The solution of claim 13, wherein the protein is whey or lactalbumin.

17. The solution of claim 12, further comprising sodium, chloride, bicarbonate, lactose, calcium and/or magnesium.

18. The solution of claim 12, wherein the pH of the solution is approximately 6.9 to 7.4.

19. A hemodialysis solution comprising amino acids, wherein the amino acids are derived from the hydrolysis of whey or lactalbumin.

20. The solution of claim 19, further comprising sodium, chloride, bicarbonate, lactose, calcium and/or magnesium.

21. The solution of claim 19, wherein the pH of the solution is approximately 6.9 to 7.4.

22. A hemodialysis solution comprising amino acids, wherein the amino acids are derived from the hydrolysis of soy protein.

23. The solution of claim 22, further comprising sodium, chloride, bicarbonate, lactose, calcium and/or magnesium.

24. The solution of claim 22, wherein the pH of the solution is approximately 6.9 to 7.4.

25-39. (canceled)
